(19) United States
(12) Patent Application Publication
(10) Pub. No.: US 2011/0159095 A1
(43) Pub. Date: Jun. 30, 2011

(54) TREATMENT OF ADRENAL INSUFFICIENCY

(76) Inventors: Hiep Huatan, Maidstone Kent (GB); Richard Ross, Sheffield (GB)

(21) Appl. No.: 13/060,781

(22) PCT Filed: Sep. 17, 2009
(86) PCT No.: PCT/GB2009/002217

§ 371 Date: Feb. 25, 2011

Related U.S. Application Data
(60) Provisional application No. 61/099,428, filed on Sep. 23, 2008.

(30) Foreign Application Priority Data
Sep. 21, 2008 (GB) 0817120.9

(51) Int. Cl. A61K 9/24; A61K 9/00; A61K 31/56

(52) U.S. Cl. 424/472; 424/400; 514/179

(57) ABSTRACT

The disclosure relates to a pharmaceutical formulation, for example a tablet, adapted for delayed and sustained release of hydrocortisone and a treatment regime that uses said tablet in the treatment of adrenal insufficiency.

The invention relates to a pharmaceutical formulation adapted for delayed and sustained release of hydrocortisone and a treatment regime that uses said formulation in the treatment of adrenal insufficiency.

In mammals the principle oscillator of circadian rhythms or central clock is in the suprachiasmatic nucleus. This master oscillator is responsible for the sleep-wake cycle and hormonal rhythms (e.g. cortisol and melatonin). It is recognised that peripheral tissues such as immune cells and the liver also have clock genes and their own molecular oscillating ability. A circadian rhythm is an internal daily biological clock which typically oscillates with a 24 hour periodicity. It is recognized that diseases or conditions are co-ordinated with biological rhythms. For example it is known that certain conditions are circadian phase dependent. Variations in pharmacokinetics have been shown for cardiovascular drugs [e.g. verapamil, enalapril], anti-asthmatics [e.g. theophylline, terbutaline, chemotherapeutic agents in the treatment of cancer, analgesics and antibiotics. In addition the dose response relationships can be modified dependent on when a drug is administered.

Controlled drug release is known in the art. Controlled drug release refers to the delivery of a drug in a specified temporal pattern. This is achieved by exploiting three features of drug release namely immediate or instantaneous drug release, delayed drug release and sustained drug release.

Immediate or instantaneous drug release results in a drug being available immediately or within a short period of time after administration to a subject. This provides a high level of control with respect to when the drug is delivered. This could be via an injection, catheter or via oral administration using capsules or tablets adapted for immediate release once swallowed. This mode of drug delivery however, can often incur undesirable consequences since for the most part it is not possible to control or modulate the drug release in line with physiological needs. In particular if a drug is to be administered in accordance with a circadian pattern and it requires dosing in the middle of the night this can only be achieved by waking the subject and disturbing sleep.

Delayed drug release is achieved when a drug is only made available to a subject some time after administration. This is typically achieved by combining the drug in a capsule or tablet that is coated or encapsulated with a pH sensitive substance that dissolves only when it reaches the small intestinal region of the gastrointestinal tract, thereby affording a delay period that is consistent with the rate of gut transit. A problem with delayed release formulations is that it is difficult to accurately control the delay period and the patient variation in gut transit time can be problematic.

Sustained release refers to release of a drug in a manner that the level of the drug is maintained at some level over an extended period of time. As with delayed release, sustained release can be problematic with respect to patient to patient variability and with respect to controlling the period of sustainment in a manner which provides sufficient coverage over an extended duration typically to enable once-daily dosing.

There is a need to develop alternative drug delivery means to provide controlled drug release in particular in accordance with the circadian rhythm of a patient.

Adrenal failure occurs in approximately 1/10,000 of the population. It may be due to either primary adrenal failure (e.g. Addison's disease commonly occurring following autoimmune damage to the adrenal gland or TB), or secondary adrenal failure (which occurs due to pituitary failure which may be caused by a pituitary tumour or surgery). In causes of primary adrenal failure ACTH levels from the pituitary will be high and in secondary adrenal failure ACTH levels are inappropriately low. Tertiary adrenal failure is another common cause of adrenal failure is suppression of the normal pituitary-adrenal axis by steroid therapy such as that used for chemotherapy, rheumatoid arthritis and asthma. Thus, adrenal failure is a relatively common condition and many patients have to take long-term steroid replacement therapy.

Hydrocortisone is the preferred steroid treatment for patients with adrenal failure. Hydrocortisone is the most commonly used drug as it is equivalent to cortisol, is rapidly absorbed in the small intestine and is inexpensive. Cortisol is released from the adrenal gland under the regulation of ACTH derived from the pituitary gland. There is a circadian rhythm to cortisol release with high levels first thing in the morning and very low levels around midnight. ACTH and thus cortisol levels begin to rise around 3 am and peak at 7 to 9 am gradually falling over the day to a nadir at midnight. Cortisol is a steroid hormone essential for survival especially during stress such as infection. Deficiency in cortisol results in fatigue, wasting, diarrhoea and finally death usually with an Addisonian crisis precipitated by infection.

This disclosure relates to the circadian delivery of hydrocortisone in the treatment of adrenal insufficiency to provide physiological replacement of cortisol and improve the quality of life of subjects suffering from adrenal insufficiency.

According to an aspect of the invention there is provided a pharmaceutical preparation adapted for oral administration comprising:
a core comprising at least hydrocortisone, a soluble dissolution modifying polymer and an insoluble dissolution modifying polymer to provide sustained release of hydrocortisone contained in said core;
an eroding layer contacting said core and comprising at least one enteric polymer that delays the release of hydrocortisone; and
an outer layer comprising a hydrophobic insoluble polymer that at least partially covers the eroding layer wherein said preparation is for use in the treatment of adrenal insufficiency.

In a preferred embodiment of the invention said core comprises at least 2.5 mg of hydrocortisone.

In a preferred embodiment of the invention said core comprises about 2.5-20 mg of hydrocortisone; preferably about 15 mg of hydrocortisone.

In an alternative preferred embodiment of the invention said core comprises about 10 mg of hydrocortisone.

In a preferred embodiment of the invention said soluble dissolution modifying polymer is a carbomer homopolymer.

In a further preferred embodiment of the invention said insoluble dissolution modifying polymer is ammonio methacrylate.

Preferably the ratio of soluble to insoluble dissolution modifying polymer is approximately 1:1 [w/w].

In a preferred embodiment of the invention said eroding layer comprises a mixture of co-polymers wherein said co-polymers are methacrylic acid and methyl methacrylate; preferably in a ratio of approximately 1:1.

In a preferred embodiment of the invention said hydrophobic insoluble polymer ammonio methacrylate.

According to an aspect of the invention there is provided a method for the treatment of adrenal insufficiency comprising:
i) administering at least a first tablet between 20:00 and 24:00 wherein said tablet comprises:
   a core comprising at least hydrocortisone, a soluble dissolution modifying polymer and an insoluble dissolution modifying polymer to provide sustained release of hydrocortisone contained in said core;
   an eroding layer contacting said core and comprising at least one enteric polymer that delays the release of hydrocortisone; and
   an outer layer comprising a hydrophobic insoluble polymer that at least partially covers the eroding layer;
ii) administering at least a second tablet between 06:00 and 08:00 wherein said tablet comprises:
   a core comprising at least hydrocortisone, a soluble dissolution modifying polymer and an insoluble dissolution modifying polymer to provide sustained release of hydrocortisone contained in said core;
   an eroding layer contacting said core and comprising at least one enteric polymer that delays the release of hydrocortisone; and
   an outer layer comprising a hydrophobic insoluble polymer that at least partially covers the eroding layer.

In a preferred method of the invention said first tablet comprises 10-30 mg of hydrocortisone; preferably about 15-20 mg of hydrocortisone.

In a preferred method of the invention said second tablet comprises 5-15 mg of hydrocortisone; preferably 10 mg of hydrocortisone.

In a preferred embodiment of the invention adrenal insufficiency is caused by a condition selected from the group consisting of: primary or secondary or tertiary adrenal failure, congenital adrenal hyperplasia, late-onset congenital adrenal hyperplasia, polycystic ovarian failure or Addison's disease.

In a preferred embodiment of the invention adrenal dysfunction is caused by congenital adrenal dysfunction.

When administered the hydrocortisone formulation is administered in pharmaceutically acceptable preparations. Such preparations may routinely contain pharmaceutically acceptable concentrations of salt, buffering agents, preservatives and compatible carriers.

The hydrocortisone is administered in effective amounts. An “effective amount” is that amount of hydrocortisone that alone, or together with further doses, produces the desired response. The dose of hydrocortisone administered to an individual patient will depend on the age, weight, BMI and body surface area of the patient. Generally very young children require a higher dose for body surface area than adults but dose is also related to weight, BMI and body surface area. Tablets will be in dose forms of 2.5 mg, 5 mg, 10 mg and 15 mg allowing for different combinations to provide a variety of twice daily treatment regimens. Thus, in a child they may receive from 2.5 to 10 mg at night and 2.5 to 5 mg in the morning whereas an adult would be expected to receive from 10 to 30 mg at night and 5 to 15 mg in the morning. These factors are well known to those of ordinary skill in the art and can be addressed with no more than routine experimentation. It is generally preferred that a maximum dose of the individual components or combinations thereof be used, that is, the highest safe dose according to sound medical judgment.

The hydrocortisone preparation used contains an effective amount of hydrocortisone for producing the desired response in a unit of weight or volume suitable for administration to a patient.

The doses of hydrocortisone administered to a subject can be chosen in accordance with different parameters, in particular the state of the subject. Other factors include the desired period of treatment. In the event that a response in a subject is insufficient at the initial doses applied, higher doses (or effectively higher doses by a different, more localized delivery route) may be employed to the extent that patient tolerance permits.

When administered, the hydrocortisone preparation is administered in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Hydrocortisone preparations may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with hydrocortisone, and with each other, in a manner such that there is no interaction which would substantially impair the desired pharmaceutical efficacy.

The formulations may include: (a) fillers such as lactose, manitose, dicalcium phosphate, microcrystalline cellulose, starch, pre-gelatanised starch, (b) binders such as hydroxypropyl cellulose, polyvinyl pyrrolidone, polyvinyl acetate, (c) powder flow enhancers such colloidal silicon dioxide (d) lubricants such as magnesium stearate, sodium stearyl fumarate (e) disintegrants such as sodium starch glycollate and polyvinyl pyrrolidone and (f) anti-sticking agents such as talc

The hydrocortisone preparation may contain suitable buffering agents, including: acetic acid in a salt; citric acid in a salt; boric acid in a salt; and phosphoric acid in a salt. The hydrophobic drug preparation also may contain, optionally, compatible preservatives or chemical and physical stabilising agents known to those skilled in the art.

According to an aspect of the invention there is provided a tablet comprising:
a core comprising 2.5-20 mg of hydrocortisone, a soluble dissolution modifying polymer and an insoluble dissolution modifying polymer to provide sustained release of hydrocortisone;
an eroding layer contacting said core and comprising an enteric polymer that delays the release of hydrocortisone; and
an outer layer comprising a hydrophobic insoluble polymer that at least partially covers the surface of the tablet.

In a preferred embodiment of the invention said tablet comprises 10-20 mg of hydrocortisone.

In an alternative preferred embodiment of the invention said tablet comprises 5-15 mg of hydrocortisone.

According to a further aspect of the invention there is provided a kit comprising:
i) a first tablet comprising:
   a core comprising 2.5-20 mg of hydrocortisone, a soluble dissolution controlling polymer and an insoluble dissolution polymer to provide sustained release of hydrocortisone;
   an eroding layer contacting said core and comprising an enteric polymer that delays the release of hydrocortisone; and
   an outer layer comprising a hydrophobic insoluble polymer that at least partially covers the surface of the tablet; and
ii) a second tablet comprising:
   a core comprising 2.5-15 mg of hydrocortisone, a soluble dissolution controlling polymer and an insoluble dissolution polymer to provide sustained release of hydcortisone;
   an eroding layer contacting said core and comprising an enteric polymer that delays the release of hydrocortisone; and
   an outer layer comprising a hydrophobic insoluble polymer that at least partially covers the surface of the tablet.

In a preferred embodiment of the invention said kit comprises a list of instructions relating to the administration of said tablets to a subject.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### Definitions

“Controlled release” is the drug release profile delivered by a dosage form, usually via zero-order or first order, with the objective of maintaining a level of drug in the bloodstream which is invariant with time over the dosing interval.

“Sustained release” is the drug release profile delivered by a dosage form, usually via first order or pseudo first order, with the objective of sustaining the duration of release such that the frequency of dosing is less than or equal to half that achieved with an immediate release dosage form.

“Delayed release” is the drug release profile delivered by a dosage form which is characterised by an initial period of complete absence of drug release or very low drug release, typically less than or equal to 10% of the overall dose in the dosage form, prior to the main drug release phase.

“Soluble dissolution modifying polymer” is a homopolymer or copolymer or mixtures thereof which are generally aqueous soluble and can undergo dissolution in an aqueous media to modulate the rate of release of drug encapsulated, entrapped, dispersed, aggregated or dissolved therein.

“Insoluble dissolution modifying polymer” is a homopolymer or copolymer or mixtures thereof which are generally water insoluble and can undergo dissolution usually by erosion by physical or chemical processes in an aqueous media to modulate the rate of release of drug encapsulated, entrapped, dispersed, aggregated or dissolved therein.

“hydrophobic insoluble polymer” is a homopolymer or copolymer or mixtures thereof which have low water solubility, typically <0.1 mg/ml.

“Eroding layer” is a layer within a dosage form which is designed to undergo dissolution or erosion when in contact with an aqueous media.

“Enteric polymer” is a homopolymer or copolymer or mixtures thereof that have pH dependent solubility in aqueous media characterised generally by low aqueous solubility under acidic conditions (pH1-4) and higher aqueous solubility under weakly acidic conditions and above (pH>5). The purpose is to protect the dosage from polymer dissolution mediated release in the acidic gastric environment of the gut.

An embodiment of the invention will now be described by example only and with reference to the following figures:

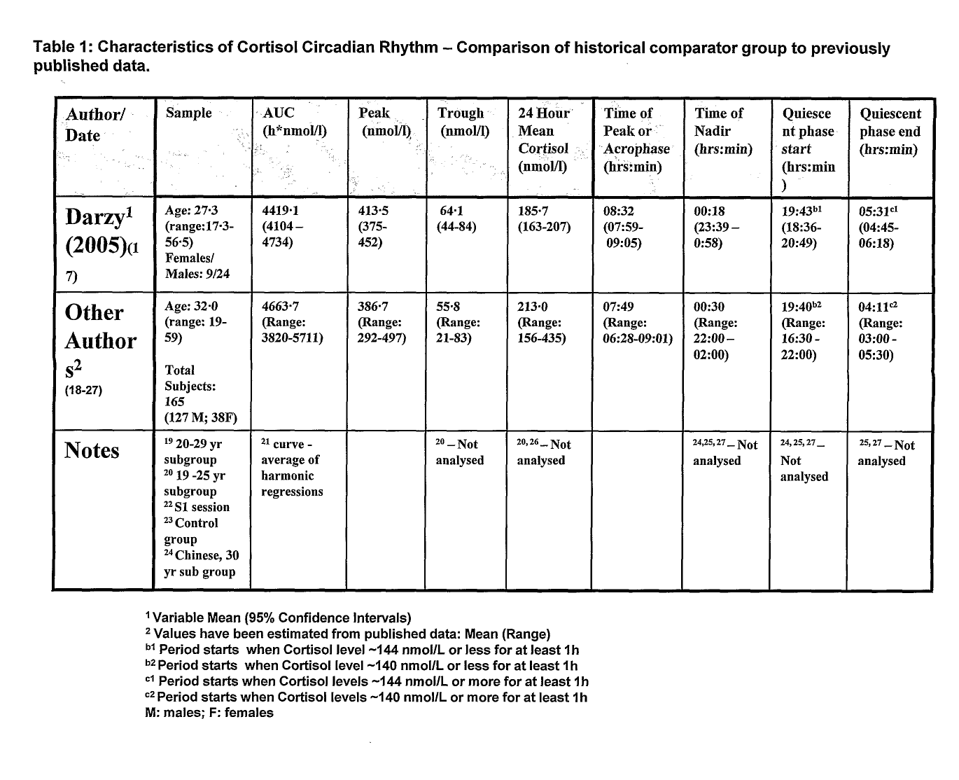

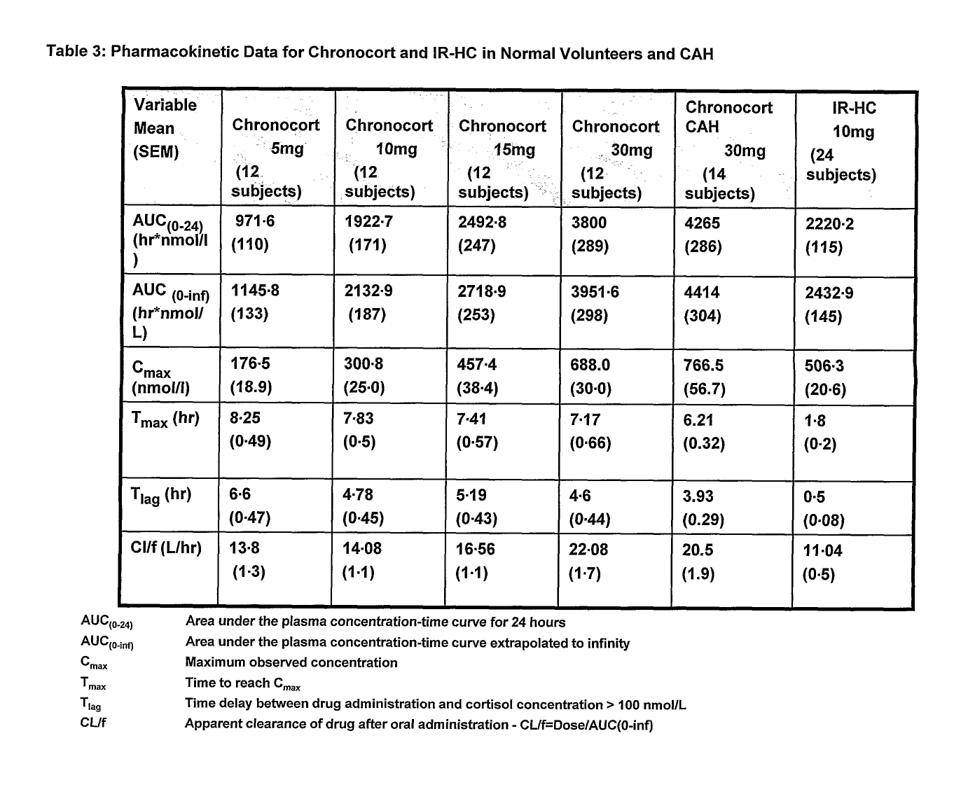

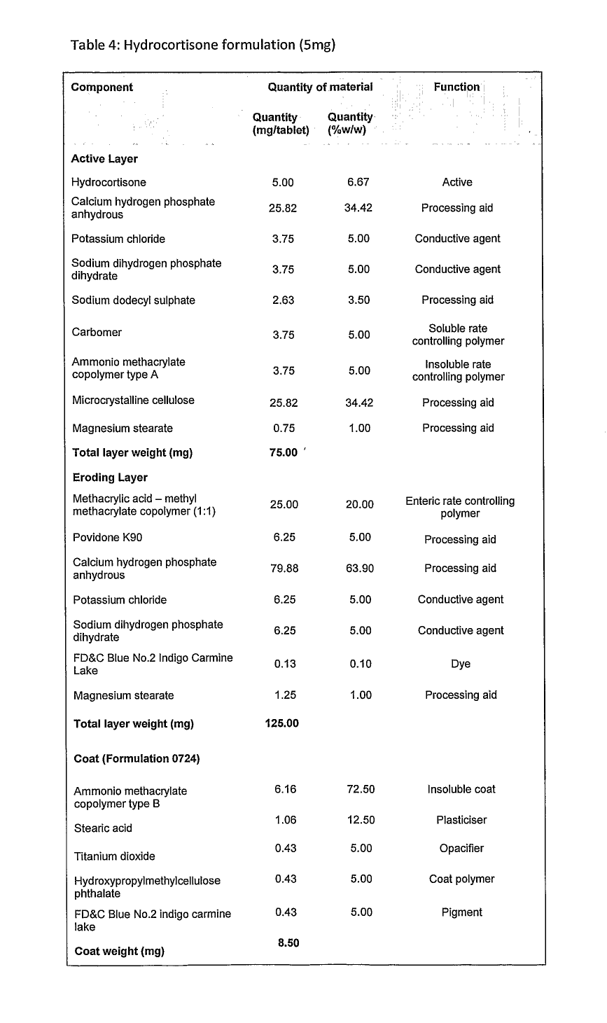

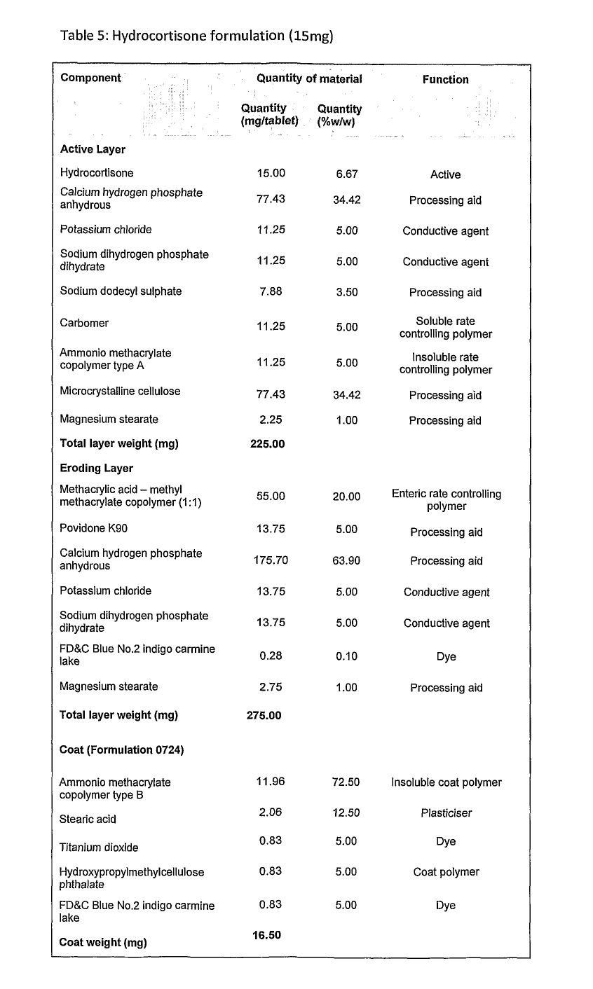

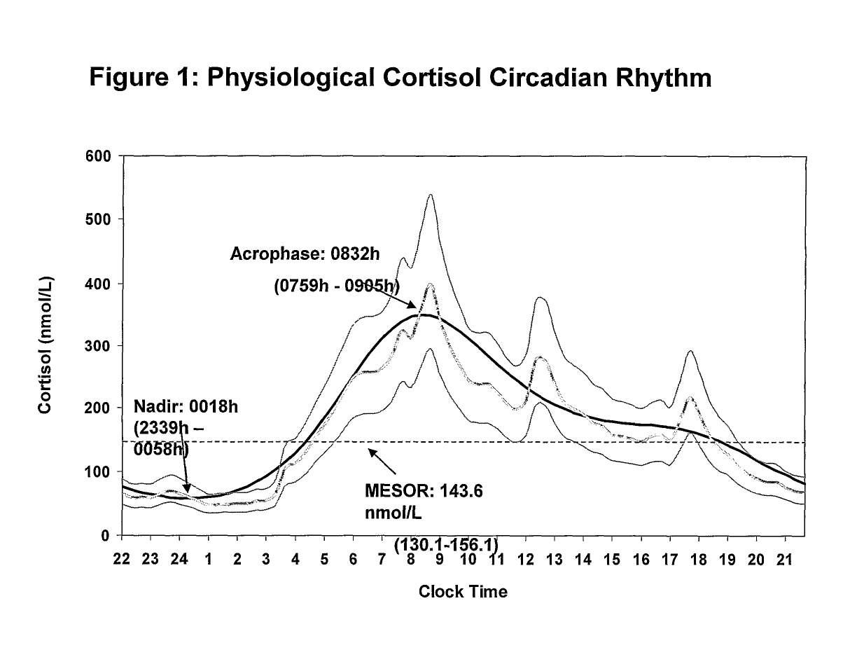

FIG. 1: Physiological Cortisol Circadian Rhythm: The figure shows the geometric mean (z,22 ) plus/minus 2SD (−) serum cortisol concentration calculated from 20-minute sampling over a 24-hour period in 33 healthy subjects. The fitted cosinor (—) is the average of harmonic regressions that were a fit for the individual subject data. Mesor=Midline estimating statistic of rhythm. Acrophase=Time of peak using a 24-hour clock with midnight taken as origin. Nadir=Time of trough cortisol level;

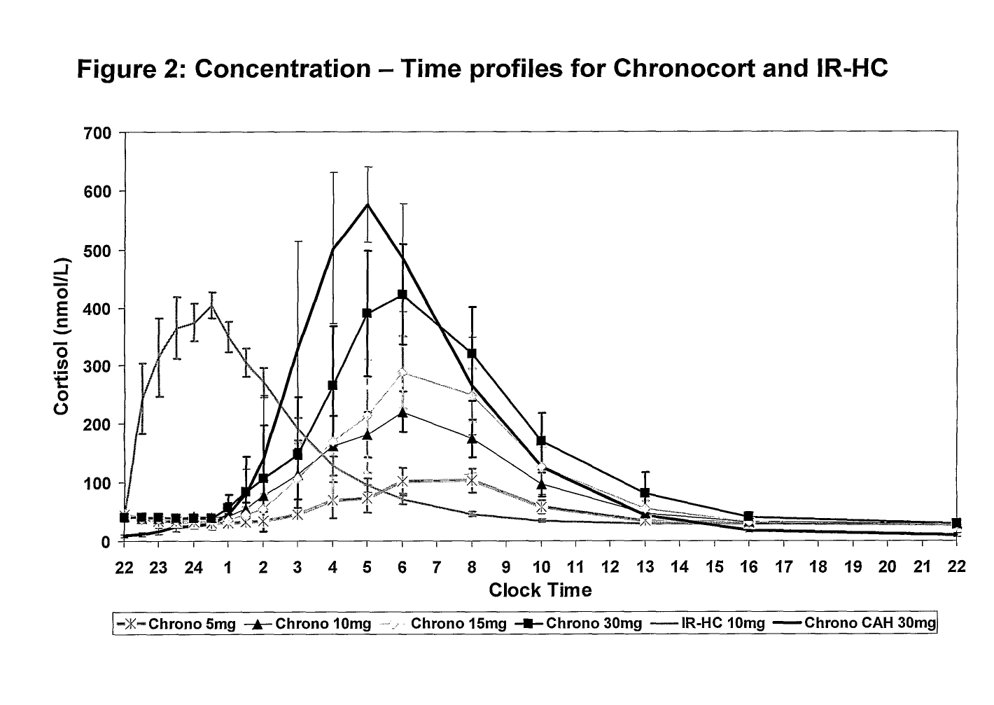

FIG. 2: Cortisol concentration-time profiles for Chronocort and IR-HC: Cortisol profiles for different doses of Chronocort given at 2200 h compared to 10 mg IR-HC using geometric means (±SEM) of serum cortisol concentrations over 24 hours in normal volunteers and CAH patients;

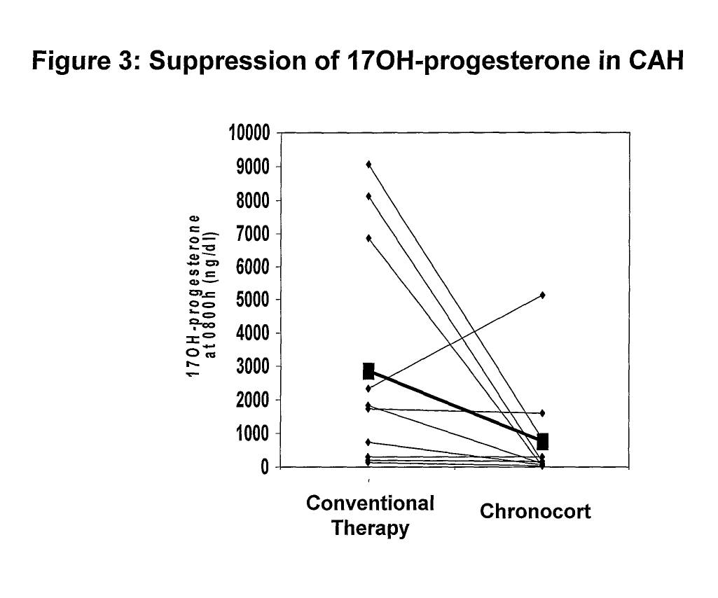

FIG. 3: 17OH-progesterone in CAH: Comparison of 0800 h 17OH-progesterone levels on conventional treatment (IR-HC, prednisone or dexamethasone) versus Chronocort. Mean levels are shown in bold; and

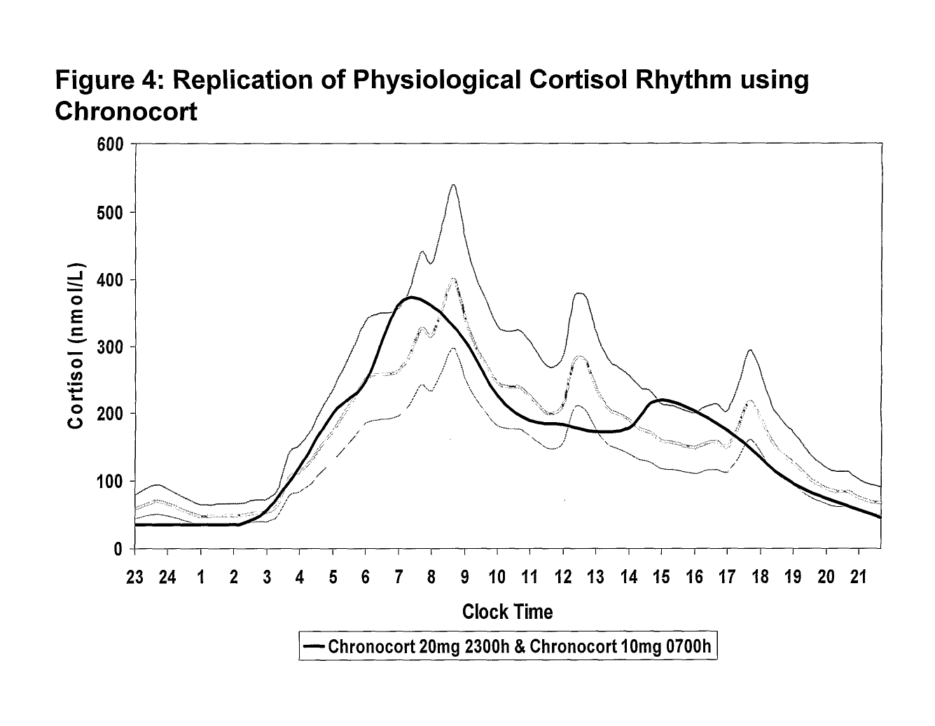

FIG. 4: Replication of Physiological Cortisol rhythm using Chronocort: The graph shows modeled concentration-time profile (—) obtained when giving 20 mg Chronocort at 2300 h and 10 mg Chronocort at 0700 h superimposed on the physiological cortisol rhythm; geometric mean ( [symbol] ) plus/minus 2SD (−).

### MATERIALS AND METHODS

Healthy reference group: 33 individuals who had undergone 24-hour, 20-minute, cortisol profiling provided data for the definition of physiological cortisol rhythm (Table 1).⁽¹⁷⁾ To further validate that this data set was representative of the general population, all variables were compared to similar variables from previously published literature.⁽¹⁸⁻²⁷⁾

Defining physiological ranges: Cortisol generally shows a skewed distribution. Therefore, values were log-transformed enabling the geometric mean to be calculated at each time point. 95% confidence intervals (CI) were calculated for: the AUC₍₀₋₂₄₎ (area under the curve from time 0 to 24 hours), peak cortisol, trough cortisol and 24-hour mean cortisol. In addition we report 95% reference ranges (±2SD) for the peak and trough cortisol.

Cosinor analysis: For each individual cortisol profile, a cosinor model with a second harmonic was fitted to the data.⁽²⁸⁾ A group cosinor model was computed by averaging the coefficients from individual fits. Circadian timing estimates were obtained for each individual profile. This allowed us to calculate the mesor (rhythm adjusted mean), the acrophase (time of peak in rhythm), the nadir (lowest point of the rhythm), and the quiescent phase (start, taken as the time when cortisol was less than or equal to the mesor for more than one hour, and end, when cortisol was greater than or equal to the mesor for more than one hour).

Pharmacokinetic Analysis for Chronocort: 32 healthy male subjects, 18 to 50 years, with no illness, operation or steroid use in the previous three months and no regular medication were recruited. The study was approved by the Plymouth Independent Ethics Committee, UK and all subjects gave informed written consent. The sampling was performed in the Chiltern Clinical Research Unit, UK.

Dose-response study: 20 subjects were randomized to receive three of the following four single-dose regimes: 5 mg Chronocort (1×5 mg), 15 mg Chronocort (1×15 mg), 30 mg Chronocort (2×15 mg), 10 mg immediate release hydrocortisone (IR-HC) with a one week washout between treatments. 12 other subjects were randomized to receive either 10 mg (2×5 mg) of Chronocort or 10 mg IR-HC with a one-week washout period. The Chronocort or IR-HC dose was taken at 2200 h. Participants had their HPA axis suppressed with 1 mg oral dexamethasone at 1800 h and 2200 h on Day 1 and then at 0600 h, 1200 h and 1800 h on Day 2 during each treatment period. Plasma ACTH levels were taken at 2155 h on Day 1 and on 0600 h on Day 2 whilst serum cortisol levels were taken prior to ingestion of the drug and were repeated every 30 minutes for the first four hours, then at 0200 h, 0300 h, 0400 h, 0500 h, 0600 h, 0800 h, 1000 h, 1300 h, 1600 h, 2200 h.

Proof of concept study in CAH: 14 patients, 7 males, 17 to 55 years, with classic CAH were studied. Inclusion criteria: CAH based on hormonal and/or genetic testing, age >16 years, detectable 17OH-progesterone, and plasma renin<1.5×the normal range measured within 8 weeks of study entry. Exclusion criteria: medication that interferes with glucocorticoid metabolism including oestrogens, co-morbidity, pregnancy and lactation. One week prior to admission to the National Institutes of Health Clinical Center (Bethesda, Md., USA), patients were switched from their usual glucocorticoid medication to thrice daily IR-HC. On admission, patients received IR-HC at 0800 only; they did not receive their 1500 dose to allow for a 14 hour washout period prior to receiving Chronocort 30 mg at 2200. Following the administration of Chronocort they underwent a 24 hour pharmacokinetic profile for cortisol. In addition, 17OH progesterone was measured at 0800 h. The study was approved by the Eunice Kennedy Shriver National Institute of Child Health & Human Development Institutional Review Board, and all subjects gave informed written consent.

Chronocort: The tablet has an insoluble barrier coat protecting all but the upper face of the tablet. The unprotected face exposes a delaying layer that slowly erodes in the small intestine to present the sustained release drug-containing layer.

The modified release hydrocortisone formulations used this clinical study are presented in two dose strengths: 5 and 15 mg per tablet.

Each tablet comprises two layers: one of which is an eroding layer conferring delayed release properties and the other is an active layer containing hydrocortisone in a sustained release matrix. An outer water-insoluble coat encapsulates the tablet core to prevent water ingress on all except the eroding layer surface. For the 15 mg formulation, the weight ratio of the eroding layer to active layer is about 1.2 and for the 5 mg formulation the weight ratio is about 1.7.

The eroding layer contains principally a mixture of erodible co-polymer (Methacrylic acid—methyl methacrylate copolymer (1:1)) and an insoluble filler calcium hydrogen phosphate anhydrous in a ratio of approximately 1:3. This mixture makes up about 84% of the eroding layer by weight. Other diluents and fillers present in the eroding layer include povidone binder, magnesium stearate, FD&C blue dye and inorganic salts to impart enhanced processing properties.

The active layer contains 5 or 15 mg hydrocortisone (present as the free base) in combination with a 1:1 mixture of two rate controlling polymers:carbomer and ammonio methacrylate copolymer type A. The rate controlling polymer mixture constitutes about 10% of the active layer by weight. The principal filler materials used in the active layer include microcrystalline cellulose and calcium hydrogen phosphate anhydrous. These materials constitute about 70% of the active layer by weight. Other diluents and fillers present in the active layer include sodium dodecyl sulphate, magnesium stearate and inorganic salts to impart enhanced processing properties.

The outer insoluble coat contains principally a water insoluble polymer: Ammonio methacrylate copolymer type B, which is present at about 72% by weight. Other components of the water insoluble coat include a plasticiser, Hydroxypropyl-methylcellulose phthalate (5% by weight), a polymer softener, stearic acid (12.5% by weight), colourant and opacifier. The coat comprises about 3-4% of the tablet by weight for both the 15 mg and 5 mg formulations.

IR-HC: 10 mg tablets from MSD (Hertfordshire, UK) were used in the healthy volunteer study and 5 and 10 mg tablets of Cortef (Pfizer, USA) in the CAH patients. In the pharmacokinetic comparison we used a 10 mg dose to avoid un-physiological peak values that exceed the binding capacity of cortisol binding globulin (CBG).

Assays: Serum cortisol was measured using the Bayer Advia Centaur Automated Immunoassay System. The inter-assay coefficient variation was 7% at 200 nmol/l (7.2 μg/dl), and 8% at 1,050 nmol/l (38 μg/dl). Plasma ACTH was measured using the DPC Immulite 2000 assay. Plasma 17OH-progesterone was measured using Liquid Chromatography Tandem Mass Spectrometry at Quest Diagnostics (Nichols Institute, San Juan Capistrano, Calif., USA) with an inter-assay coefficient of variation of 8.2%.

Statistical Analysis: Pharmacokinetic (PK) parameters were computed by non-compartmental analysis. As only a 10 mg dose of IR-HC was used, Chronocort was dose adjusted as follows: (AUC(Chronocort)*Dose(IR-HC)/(AUC(IR-HC)*Dose(Chronocort). Dose proportionality was determined by comparing AUC_{(0-inf)}, and Cₘₐₓ of the 5 mg, 15 mg and 30 mg Chronocort doses using a linear model approach on the log-transformed pharmacokinetic parameters versus log-transformed doses. Slope estimates and corresponding 90% Cls were calculated. Dose proportionality was assumed if the slope was not statistically significantly (P>0.1) different from unity. In modeling we simulated giving Chronocort doses at different times and once or twice daily to establish whether Chronocort could produce a 24-hour physiological cortisol profile. To examine for the best fit we calculated the ratio between the AUC of Chronocort versus the AUC for the physiological cortisol profile at each of the pharmacokinetically relevant time intervals, e.g. the trapezoidal segment between each sampling interval. We determined what proportion of Chronocort cortisol levels would fall between the upper and lower 95% reference ranges for physiological cortisol levels. A ratio between 0.8 and 1.2 (within 20% of identity) was considered an acceptable Chronocort fit. For comparison of 0800 17OH-progesterone in CAH patients the data was log transformed and compared by paired t-Test.

### EXAMPLE 1

Defining the Physiological Cortisol Circadian Rhythm: A circadian rhythm is clearly demonstrated in the healthy group (FIG. 1). A cosinor model with second harmonic gave an excellent fit (r²=0.97, p<0.001). All individuals had a significant (p<0.001) sinusoidal rhythm. Cortisol reached a peak at 0832 h (95% Cl 0759 h-0905 h), then gradually decreased until reaching a nadir at 0018 h (95% Cl 2339-0058 h). Two smaller peaks occurred at meal times. All of the reference group means obtained were similar to previously published literature (Table 1).⁽¹⁸⁻²⁷⁾

### EXAMPLE 2

Pharmacokinetic Analysis of Chronocort: Four of the 32 participants were withdrawn; two for protocol violations, one withdrew consent and one had an adverse event whilst on IR-HC (hiccups). Twenty eight subjects completed the study (Table 2). 98% of all ACTH values were below the assay lower limit confirming dexamethasone-induced suppression of HPA axis. The cortisol profiles after Chronocort and IR-HC are shown in FIG. 2. Chronocort formulations showed a marked prolongation in Tₘₐₓ, T_{lag} and a lower dose-adjusted Cₘₐₓ compared to hydrocortisone (Table 3), highlighting the characteristics of a delayed and sustained release formulation. The mean (90% Cl) relative bioavailability for Chronocort to IR-HC was: 100% (90-112%) for 5 mg, 79% (66-95%) for 10 mg, 86% (77-96%) for 15 mg, and 69% (62-77%) for 30 mg. The peak concentrations and cortisol exposure increased predictably with increasing Chronocort doses. Dose proportionality for AUC_{(0-inf)} and Cₘₐₓ(slope (90% Cls)) was observed between Chronocort 5 mg and 15 mg: 0.82(0.62-1.02) and 0.9(0.69-1.1), respectively, and was not observed between Chronocort 15 mg and 30 mg: 0.58(0.30-0.85) and 0.64(0.40-0.87).

### EXAMPLE 3

Comparison of Chronocort to the Physiological Cortisol: Compared to the physiological profile, 30 mg Chronocort provided the best cortisol exposure over 24 hours (AUC mean (90% Cl): 88% (77-99%)); however, Cₘₐₓ was higher (mean±SEM: 688±30 vs 400±22 nmol/l) and the peak occurred earlier (mean: 0600 h vs 0832 h). The Chronocort cortisol profile suggests that it provides approximately 12 hours exposure to hydrocortisone (FIG. 2). Compared to the physiological profile, cortisol AUC for 12 hours after 2200 h for 15 mg Chronocort was 84% (71-97%), the Cₘₐₓ was similar (457±38 vs 400±22 nmol/l) but occurred earlier (0600 h vs 0832 h).

### EXAMPLE 4

Proof of concept in CAH: Patients were on a variety of treatment regimens that included: dexamethasone at night or twice daily (n=8, dose 0.25 to 0.75 mg), prednisone twice daily (n=2, dose 3.5 to 6.0 mg) and IR-HC thrice daily (n=4, dose 25 to 40 mg). The cortisol profile and pharmacokinetics of Chronocort 30 mg in patients with CAH were similar to those seen in healthy volunteers (Table 3, FIG. 2). When omitting two patients on dexamethasone one who had very low baseline 17OH-progesterone (<100 ng/dl) and the other with missing data, patients had a significantly lower 17OH-progesterone on Chronocort versus conventional therapy: (mean±SEM: 770±461 vs 2859±1037 ng/dl, p=0.01). Only one patient had a much higher 17OH-progesterone whilst on Chronocort compared to conventional treatment. This patient was on dexamethasone 0.5 mg at night.

### EXAMPLE 5

Modeling Chronocort Data to provide physiological dosing regimen: For Chronocort 30 mg given at 2200 h, only 2 cortisol AUCs of the 16 (12.5%) fell within the 0.8 to 1.2 ratio. However, 12 (75%) of 16 cortisol AUCs fell within the 0.8 to 1.2 ratio for Chronocort given as 20 mg at 2300 h and 10 mg at 0700. Using this analysis we defined the best dose combination to provide physiological cortisol levels as either 15 or 20 mg Chronocort at 2300 h and 10 mg Chronocort at 0700 h (FIG. 4).

### EXAMPLE 6

Tables 4 and 5 exemplify the composition for hydrocortisone formulations capable of providing delayed and sustained release functionality for use in the treatment of adrenal insufficiency and related diseases. In the formulation examples provided, hydrocortisone is formulated at 5 mg (Table 4) and at 15 mg (Table 5).

In Tables 4 and 5, the tablet core formulation typically comprises at least hydrocortisone, a water soluble dissolution modifying polymer e.g., carbomer, ammonio methacrylate copolymer type B (Eudragit® RS100) and an insoluble dissolution modifying polymer e.g., ammonio methacrylate copolymer type A (Eudragit® RL100). The core is typically attached or adjacent to an eroding layer which comprises at least one enteric polymer e.g., methacrylic acid-methyl methacrylate copolymer (1:1) (Eudragit® L100) that delays the release of hydrocortisone. The core and the eroding layer is further surrounded by an outer layer which partially covers the eroding layer and comprises a hydrophobic water insoluble polymer e.g., ammonio methacrylate copolymer type B (Eudragit® RS100). Excipients which aid the processing and tabletting properties of hydrocortisone and formulation therein are typically included in the core and eroding layer composition e.g., calcium hydrogen phosphate anhydrous, microcrystalline cellulose, magnesium stearate, povidone K90, sodium dodecyl sulphate and identification dye (FD&C). Furthermore, certain conductive agents are also included if electrostatic coating processes are used to deposit the outer water insoluble polymer layer e.g., potassium chloride, sodium dihydrogen phosphate dihydrate. Plasticisers such as hypromellose phthalate are also used in the outer layer to impart processability. The formulations exemplified in Tables 4 and 5 are prepared using the following steps:

### Description of the Manufacturing Process for Formulations Described in Tables 4 and 5

Preparation of the Core Layer
1. Blend Hydrocortisone, carbomer, ammonio methacrylate copolymer type A, dibasic calcium phosphate, anhydrous, microcrystalline cellulose and sodium dodecyl sulphate in a planetary mixer.
2. Dissolve potassium chloride and monobasic sodium phosphate, dihydrate in the specified amount of purified water and use to granulate the blend. Granulate to visual end-point with additional purified water as required.
3. Dry the granulate to a moisture content of ≦2%.
4. Screen the granule to <1 mm.
5. Pass the magnesium stearate through a suitable screen with an equal quantity of dried granule. Lubricate the granule with the magnesium stearate by blending.

Preparation of Eroding Layer
1. Blend methacrylic acid-methyl methacrylate copolymer (1:1), dibasic calcium phosphate anhydrous, povidone K90 and FD&C blue (lake) in a planetary mixer.
2. Dissolve potassium chloride and monobasic sodium phosphate, dihydrate in the specified amount of purified water and use to granulate the blend. Granulate to visual end-point with additional purified water as required.
3. Dry the granulate to a moisture content of <2% w/w.
4. Screen the granule to <1 mm.
5. Pass the magnesium stearate through a suitable screen with an equal quantity of dried granule. Lubricate the granule with the magnesium stearate by blending.

Tabletting
1. Set up the tablet press with 10 mm round flat bevelled punches.
2. Place the eroding layer and the core layer into the respective hoppers
3. Compress bi-layer tablets in accordance with the fill-weight specified for each formulation.

### Outer Layer Coating

Ammonio methacrylate copolymer (type B) and hypromellose phthalate (HPMCP) are blended together. Stearic acid, titanium dioxide and FD&C Blue No.2 (indigo carmine lake) are blended together. These two mixes are then transferred to a high shear mixer and blended well. The mix is then extruded using a twin screw extruder-feeder into flakes. The flakes are milled to a particle size of less than 1000 μm. Milled material is then micronized and classified to ensure a final particle size d₅₀ of typically, approximately 10 μm. Use the electrostatic dry powder deposition machine to coat bi-layer tablets over the active surface and sides, leaving the inactive eroding layer exposed. Fuse the powder coating to the tablet core at approximately 130° C. for 4 minutes.

TABLE 2
Demographics for study subjects

| | Historical | | |
|---|---|---|---|
| | Comparator | Healthy | CAH |
| | Group | Volunteers | Group |
| Number of | 33 | 28 | 14 |
| Subjects | | | |
| Sex | 24 Males | 28 Males | 7 Males |
| Age (range) | 27.3 | 32.7 | 25.8 |
| | (17-56) | (21-46) | (17-55) |
| Weight | 67.1 | 80.0 | 77.5 |
| Mean (95% CI) | (62.2-72.0) | (75.4-84.6) | (68.8-86.2) |
| BMI | 22.9 | 25.6 | 28.4 |
| Mean (95% CI) | (21.9-23.9) | (24.3-26.9) | (25.6-31.2) |

### REFERENCES

1 Krieger D T, Allen W, Rizzo F, Krieger H P. Characterization of the normal temporal pattern of plasma corticosteroid levels. Journal of Clinical Endocrinology & Metabolism. 1971; 32:266-84.

2 Balsalobre A, Damiola F, Schibler U. A serum shock induces circadian gene expression in mammalian tissue culture cells. Cell. 1998; 93:929-37.

3 Balsalobre A, Brown S A, Marcacci L, Tronche F, Kellendonk C, Reichardt H M, et al. Resetting of circadian time in peripheral tissues by glucocorticoid signaling. Science. 2000; 289:2344-7.

4 Buckley T M, Schatzberg A F. On the interactions of the hypothalamic-pituitary-adrenal (HPA) axis and sleep: normal HPA axis activity and circadian rhythm, exemplary sleep disorders. Journal of Clinical Endocrinology & Metabolism. 2005; 90:3106-14.

5 Plat L, Leproult R, L'Hermite-Baleriaux M, Fery F, Mockel J, Polonsky K S, et al. Metabolic effects of short-term elevations of plasma cortisol are more pronounced in the evening than in the morning. J Clin Endocrinol Metab. 1999; 84:3082-92.

6 Spiegel K, Leproult R, Van Cauter E. Impact of sleep debt on metabolic and endocrine function. Lancet. 1999; 354:1435-9.

7 Bergthorsdottir R, Leonsson-Zachrisson M, Oden A, Johannsson G. Premature mortality in patients with Addison's disease: a population-based study. J Clin Endocrinol Metab. 2006; 91:4849-53.

8 Tomlinson J W, Holden N, Hills R K, Wheatley K, Clayton R N, Bates A S, et al. Association between premature mortality and hypopituitarism. West Midlands Prospective Hypopituitary Study Group. Lancet. 2001; 357:425-31.

9 Mah P M, Jenkins R C, Rostami-Hodjegan A, Newell-Price J, Doane A, Ibbotson V, et al. Weight-related dosing, timing and monitoring hydrocortisone replacement therapy in patients with adrenal insufficiency. Clinical Endocrinology. 2004; 61:367-75.

10 Arlt W, Allolio B. Adrenal insufficiency. Lancet. 2003; 361:1881-93.

11 Hahner S, Loeffler M, Fassnacht M, Weismann D, Koschker A C, Quinkler M, et al. Impaired subjective health status in 256 patients with adrenal insufficiency on standard therapy based on cross-sectional analysis. J Clin Endocrinol Metab. 2007; 92:3912-22.

12 Lovas K, Loge J H, Husebye E S. Subjective health status in Norwegian patients with Addison's disease. Clinical Endocrinology. 2002; 56:581-8.

13 Merke D P, Bornstein S R. Congenital adrenal hyperplasia. Lancet. 2005; 365:2125-36.

14 Merza Z, Rostami-Hodjegan A, Memmott A, Doane A, Ibbotson V, Newell-Price J, et al. Circadian hydrocortisone infusions in patients with adrenal insufficiency and congenital adrenal hyperplasia. Clinical Endocrinology. 2006; 65:45-50.

15 Lovas K, Husebye E S. Continuous subcutaneous hydrocortisone infusion in Addison's disease. European journal of endocrinology/European Federation of Endocrine Societies. 2007; 157:109-12.

16 Newell-Price J, Whiteman M, Rostami-Hodjegan A, Darzy K, Shalet S, Tucker G T, et al. Modified-release hydrocortisone for circadian therapy: a proof-of-principle study in dexamethasone-suppressed normal volunteers. Clinical Endocrinology. 2008; 68:130-5.

17 Darzy K H, Shalet S M. Absence of adrenocorticotropin (ACTH) neurosecretory dysfunction but increased cortisol concentrations and production rates in ACTH-replete adult cancer survivors after cranial irradiation for nonpituitary brain tumors. The Journal of clinical endocrinology and metabolism. 2005; 90:5217-25.

18 Weitzman E D, Fukushima D, Nogeire C, Roffwarg H, Gallagher T F, Hellman L. Twenty-four hour pattern of the episodic secretion of cortisol in normal subjects. The Journal of clinical endocrinology and metabolism. 1971; 33:14-22.

19 Van Cauter E, Leproult R, Kupfer D J. Effects of gender and age on the levels and circadian rhythmicity of plasma cortisol. The Journal of clinical endocrinology and metabolism. 1996; 81:2468-73.

20 Sharma M, Palacios-Bois J, Schwartz G, Iskandar H, Thakur M, Quirion R, et al. Circadian rhythms of melatonin and cortisol in aging. Biological psychiatry. 1989; 25:305-19.

21 Hurwitz S, Cohen R J, Williams G H. Diurnal variation of aldosterone and plasma renin activity: timing relation to melatonin and cortisol and consistency after prolonged bed rest. J Appl Physiol. 2004; 96:1406-14.

22 Selmaoui B, Touitou Y. Reproducibility of the circadian rhythms of serum cortisol and melatonin in healthy subjects: a study of three different 24-h cycles over six weeks. Life sciences. 2003; 73:3339-49.

23 Selmaoui B, Lambrozo J, Touitou Y. Endocrine functions in young men exposed for one night to a 50-Hz magnetic field. A circadian study of pituitary, thyroid and adrenocortical hormones. Life sciences. 1997; 61:473-86.

24 Zhao Z Y, Xie Y, Fu Y R, Li Y Y, Bogdan A, Touitou Y. Circadian rhythm characteristics of serum cortisol and dehydroepiandrosterone sulfate in healthy Chinese men aged 30 to 60 years. A cross-sectional study. Steroids. 2003; 68:133-8.

25 Kanabrocki E L, Sothern R B, Scheving L E, Vesely D L, Tsai T H, Shelstad J, et al. Reference values for circadian rhythms of 98 variables in clinically healthy men in the fifth decade of life. Chronobiology international. 1990; 7:445-61.

26 Kronfol Z, Nair M, Zhang Q, Hill E E, Brown M B. Circadian immune measures in healthy volunteers: relationship to hypothalamic-pituitary-adrenal axis hormones and sympathetic neurotransmitters. Psychosomatic medicine. 1997; 59:42-50.

27 Reinberg A, Lagoguey M, Cesselin F, Touitou Y, Legrand J C, Delassalle A, et al. Circadian and circannual rhythms in plasma hormones and other variables of five healthy young human males. Acta endocrinologica. 1978; 88:417-27.

28 Chakraborty A, Krzyzanski W, Jusko W J. Mathematical modeling of circadian cortisol concentrations using indirect response models: comparison of several methods. Journal of pharmacokinetics and biopharmaceutics. 1999; 27:23-43.

29 Charmandari E, Johnston A, Brook C G, Hindmarsh P C. Bioavailability of oral hydrocortisone in patients with congenital adrenal hyperplasia due to 21-hydroxylase deficiency. Journal of Endocrinology. 2001; 169:65-70.

30 Merke D P. Approach to the adult with congenital adrenal hyperplasia due to 21-hydroxylase deficiency. The Journal of clinical endocrinology and metabolism. 2008; 93:653-60.

1. A pharmaceutical preparation adapted for oral administration comprising:
a core comprising at least hydrocortisone, a soluble dissolution modifying polymer and an insoluble dissolution modifying polymer to provide sustained release of hydrocortisone contained in said core;
an eroding layer contacting said core and comprising at least one enteric polymer that delays the release of hydrocortisone; and
an outer layer comprising a hydrophobic insoluble polymer that at least partially covers the eroding layer wherein said preparation is for use in the treatment of adrenal insufficiency.

2. The preparation according to claim 1 wherein said core comprises at least 5 mg hydrocortisone.

3. The preparation according to claim 1 wherein said core comprises about 2.5-20 mg of hydrocortisone.

4. The preparation according to claim 1, wherein said soluble dissolution modifying polymer is a carbomer homopolymer.

5. The preparation according to claim 4, wherein said insoluble dissolution modifying polymer is ammonio methacrylate.

6. The preparation according to claim 1, wherein the ratio of soluble to insoluble dissolution modifying polymer is approximately 1:1 [w/w].

7. A The preparation according to claim 1, wherein said eroding layer comprises a mixture of co-polymers wherein said co-polymers are methacrylic acid and methyl methacrylate.

8. A The preparation according to claim 1, wherein said hydrophobic insoluble polymer ammonio methacrylate.

9. A method for the treatment of adrenal insufficiency comprising:
i) administering at least a first tablet between 20:00 and 24:00 wherein said tablet comprises:
a core comprising at least hydrocortisone, a soluble dissolution modifying polymer and an insoluble dissolution modifying polymer to provide sustained release of hydrocortisone contained in said core;
an eroding layer contacting said core and comprising at least one enteric polymer that delays the release of hydrocortisone; and
an outer layer comprising a hydrophobic insoluble polymer that at least partially covers the eroding layer; and
ii) administering at least a second tablet between 06:00 and 08:00 wherein said tablet comprises:
a core comprising at least hydrocortisone, a soluble dissolution modifying polymer and an insoluble dissolution modifying polymer to provide sustained release of hydrocortisone contained in said core;
an eroding layer contacting said core and comprising at least one enteric polymer that delays the release of hydrocortisone; and
an outer layer comprising a hydrophobic insoluble polymer that at least partially covers the eroding layer.

10. The method according to claim 9 wherein said first tablet comprises 10-20 mg of hydrocortisone.

11. The method according to claim 9, wherein said second tablet comprises 2.5-15 mg of hydrocortisone.

12. The method according to claim 9 wherein adrenal insufficiency is caused by primary or secondary or tertiary adrenal failure, congenital adrenal hyperplasia, late-onset congenital adrenal hyperplasia, polycystic ovarian failure or Addison's disease.

13. The method according to claim 12 wherein adrenal dysfunction is caused by congenital adrenal dysfunction.

14. A tablet comprising:
a core comprising 2.5-20 mg of hydrocortisone, a soluble dissolution modifying polymer and an insoluble dissolution modifying polymer to provide sustained release of hydrocortisone;
an eroding layer contacting said core and comprising an enteric polymer that delays the release of hydrocortisone; and
an outer layer comprising a hydrophobic insoluble polymer that at least partially covers the surface of the tablet.

15. The tablet according to claim 14, wherein said tablet comprises 10-20 mg of hydrocortisone.

16. The tablet according to claim 14, wherein said tablet comprises 2.5-15 mg of hydrocortisone.

17. A kit comprising:
i) a first tablet comprising:
a core comprising 10-20 mg hydrocortisone, a soluble dissolution controlling polymer and an insoluble dissolution polymer to provide sustained release of hydcortisone;
an eroding layer contacting said core and comprising an enteric polymer that delays the release of hydrocortisone; and
an outer layer comprising a hydrophobic insoluble polymer that at least partially covers the surface of the tablet; and
ii) a second tablet comprising:
a core comprising 2.5-15 mg hydrocortisone, a soluble dissolution controlling polymer and an insoluble dissolution polymer to provide sustained release of hydcortisone;
an eroding layer contacting said core and comprising an enteric polymer that delays the release of hydrocortisone; and
an outer layer comprising a hydrophobic insoluble polymer that at least partially covers the surface of the tablet.

18. A The kit according to claim 17, wherein said kit comprises a list of instructions relating to the administration of said tablets to a subject.

19. The preparation according to claim 1, wherein said core comprises about 10 mg or 15 mg of hydrocortisone.
